(19) United States
(12) Patent Application Publication
(10) Pub. No.: US 2022/0379040 A1
(43) Pub. Date: Dec. 1, 2022

(54) DOSE SENSING MODULE WITH FRICTION ENHANCING MEANS

(71) Applicant: Novo Nordisk A/S, Bagsvaerd (DK)

(72) Inventors: Janus Helbo, Koebenhavn N (DK); Nikolaj Eusebius Jakobsen, Soeborg (DK)

(73) Assignee: Novo Nordisk A/S, Bagsvaerd (DK)

(21) Appl. No.: 17/761,610

(22) PCT Filed: Aug. 26, 2020
(86) PCT No.: PCT/EP2020/073871

§ 371 Date: Mar. 18, 2022

(30) Foreign Application Priority Data
Oct. 8, 2019 (EP) 19201824.0

(51) Int. Cl. A61M 5/315

(52) U.S. Cl. CPC A61M 5/31568; A61M 5/31515

(57) ABSTRACT

The present invention provides a sensor module adapted to be arranged in a cartridge based drug delivery device between a rotatable piston rod and a cartridge piston for capturing dose data from a dose expelling event, the sensor module (50) extending along a reference axis and comprising: a first module part (51) adapted to engage the cartridge piston and comprising anti-rotation means for establishing a frictional interface to the cartridge to impede rotation of the first module part (51) relative thereto, a second module part (54) adapted to engage the rotatable piston rod, and sensor means (52, 152, 252; 53, 153, 253) adapted to detect an extent of relative rotational motion between the first module part (51) and the second module part (54), wherein the anti-rotation means comprises a plurality of radially outwardly projecting studs (51.1; 351.1; 451.1), each comprising a contact surface (51.8; 351.8; 451.8, 451.9) adapted to establish frictional contact with an interior surface of the cartridge.

### FIELD OF THE INVENTION

The present invention relates to rotary encoders for use in drug delivery devices and to drug delivery devices employing rotary encoders for automatically capturing an amount of drug expelled from a drug reservoir.

### BACKGROUND OF THE INVENTION

Injection devices, such as injection pens, are widely used for self-administration of liquid drugs by people in need of therapeutic treatment. Many injection devices are capable of repeatedly setting and injecting either a fixed or a variable volume of drug upon operation of respective dose setting and dose expelling mechanisms in the device. Some injection devices are adapted to be loaded with a prefilled drug reservoir containing a volume of drug which is sufficient to provide for a number of injectable doses. When the reservoir is empty, the user replaces it with a new one and the injection device can thus be used again and again. Other injection devices are prefilled when delivered to the user and can only be used until the drug reservoir has been emptied, after which the whole injection device is discarded. The various injection devices typically expel the drug by advancing a piston in the reservoir using a motion-controlled piston rod.

Within some therapy areas the tendency of a patient to adhere to the prescribed therapy is dependent on the simplicity of the specific treatment regimen. For example, many people with type 2 diabetes are diagnosed with the disease at a relatively high age where they are less prone to accept a treatment that intervenes too much with their normal way of living. Most of these people do not like to be constantly reminded of their disease and, consequently, they do not want to be entangled in complex treatment patterns or waste time on learning to operate cumbersome delivery systems. In essence, many are of the opinion that the less manual involvement the better.

For a person with diabetes it is important to timely administer one or more glucose regulating agents to maximise the time spent in normoglycemia. In that connection, in order to establish an overview of one's adherence to a particular treatment regimen, it is significant to keep track of both when such a regulating agent is administered and how much is administered. Accordingly, it is recommended that the person keeps a log of administered dose sizes and times of administration.

Previously, the establishment and maintenance of such a log would require manually noting down the data, e.g. on paper or a pc. However, as this would entail frequent active involvement many people neglected the importance of establishing the overview. In recognition of this undesirable situation various solutions have been suggested for automatic capturing of the relevant information from the individual injection devices.

For example, WO 2018/078178 (Novo Nordisk A/S) discloses a pen type injection device having a sensor arranged on a deflectable exterior surface of the injection device housing. The deflectable exterior surface is configured to undergo a deflection at a specific angular displacement of an interior component rotationally locked to the piston rod, and the sensor is adapted to output a signal in response to a detected deflection, the signal thus being representative of the angular displacement of the piston rod. Since the amount of drug expelled by the disclosed injection device correlates with the total angular displacement of the piston rod relative to the housing the output signals are automatically captured by a processor in the injection device and used as a basis for an estimation of the administered dose. In addition, the processor may establish a time for reception of the output signals and provide a time stamp for the dose expelling event. The data may then be retrieved via an electronic display on the injection device or by wireless transmission to an external device e.g. having, or being connectable to, a display.

An alternative dose detection solution is presented in WO 2014/128155 (Novo Nordisk A/S) which discloses a pen-type drug delivery device with a fully integrated sensor unit in the form of a piston washer module arranged between the piston rod of the dose expelling mechanism and the cartridge piston. The sensor unit operates like a rotary encoder and comprises a first sensor part which is engaged with the piston rod and a second sensor part which is engaged with the cartridge piston. The relative angular displacement between the two sensor parts exhibited during a dose expelling event, when the piston rod rotates relative to the drug delivery device housing and the cartridge, is detected galvanically and translated to an estimate of the size of the administered dose.

In the case of the latter sensor principle it is important for the accuracy of the dose estimation that the detected relative angular displacement between the sensor parts reflects the total angular displacement of the piston rod relative to a stationary reference, such as the drug delivery device housing or the cartridge. It is thus important to limit any potential rotation of the cartridge piston as it advances into the cartridge during the dose expelling. Such rotation could occur as a result of a transmission of the torque from the rotating piston rod internally in the sensor from the first sensor part to the second sensor part.

### SUMMARY OF THE INVENTION

It is an object of the invention to eliminate or reduce at least one drawback of the prior art, or to provide a useful alternative to prior art solutions.

In particular, it is an object of the invention to provide a rotary encoder based dose sensing module for use in a drug delivery device to automatically and with high accuracy capture information regarding a delivered dose.

It is another object of the invention to provide such a dose sensing module which does not significantly affect the force required to advance an actuator of the drug delivery device.

It is a further object of the invention to provide a drug delivery device with such a dose sensing module.

It is a further object of the invention to provide a drug delivery system comprising the drug delivery device and the dose sensing module, which drug delivery system can be stored and transported in a safe pre-use state where there is no risk of accidentally activating the dose sensing module.

In the disclosure of the present invention, aspects and embodiments will be described which will address one or more of the above objects and/or which will address objects apparent from the following text.

In one aspect the invention provides a sensor module as defined in claim 1.

Accordingly, a sensor module for use in a cartridge based drug delivery device, such as an injection device, e.g. of the pen-shaped type, is provided. The sensor module, which extends along a reference axis, is adapted to be arranged in the drug delivery device between a rotatable piston rod and a cartridge piston such that a first module part engages the cartridge piston and a second module part engages the piston rod. Thereby, the first module part becomes rotationally locked with respect to cartridge piston and the second module part becomes rotationally locked with respect to the piston rod, and the sensor means can resultantly determine the extent of relative rotational motion between the piston rod and the cartridge piston by detecting the extent of relative rotational motion between the first module part and the second module part.

For a drug delivery device having a dose expelling mechanism of the type which involves a threadedly mounted piston rod that advances helically during dose expelling the extent of relative rotational motion between the piston rod and the cartridge piston is indicative of an expelled dose. Hence, an estimation of the size of the expelled dose can be provided based on an output from the sensor means.

An accurate dose estimation requires that the cartridge piston is advanced strictly translationally, i.e. non-rotationally, in the cartridge. This is because the axial displacement of the cartridge piston determines the volume of expelled drug, and the axial displacement of the cartridge piston is determined by the axial displacement of the piston rod relative to the cartridge which again, due to the threaded mounting of the piston rod, is determined by the angular displacement of the piston rod relative to the housing. A correct determination of the axial displacement of the cartridge piston is thus tied to a correct determination of the angular displacement of the piston rod relative to a rotationally fixed reference.

In order to impede rotation of the first module part relative to the cartridge, the first module part is provided with anti-rotation means for securing a frictional interface to the cartridge. The present inventors have found that anti-rotation means comprising a plurality of radially outwardly projecting studs, each stud comprising a contact surface adapted to establish frictional contact with an interior surface of the cartridge, offers a particularly attractive compromise in relation to the conflicting desires to maximise friction to avoid rotation and at the same time minimise friction to reduce the injection force needed to axially displace the cartridge piston along the interior surface of the cartridge. If the friction is too high a dose expelling may be prevented from being carried out in the first place or may at least require an unattractively high injection force. For example, in case of a conventionally dimensioned automatic injection device where the dose expelling is enabled by an internal energy source, such as a pre-strained spring, the energy available for advancing the piston rod may be insufficient if the friction exceeds a certain level.

The radially outwardly projecting studs may be circumferentially spaced apart along an annular outer surface of the first module part. In particular, the radially outwardly projecting studs may be equidistantly spaced apart to thereby obtain equally distributed contact interfaces, maximising the stability of the first module part in the cartridge.

The radial dimension of the first module part may be larger than an inner diameter of an associated cartridge, and the contact surfaces, or at least a subset thereof, may be radially inwardly displaceable against a bias force to thereby individually apply a radially outwardly directed force to the interior surface of the cartridge, sporadically enhancing the frictional interface.

A conventional cartridge comprises a cylindrical main body with a distal shoulder and neck section bridging to an outlet end, which is sealed by a penetrable self-sealing septum, and a proximal open end which has a small circumferential bead, providing a somewhat narrowed rear entrance section. A slidable piston is arranged to seal the cartridge proximally such that an exterior cartridge cavity is formed by a proximal end portion of the cylindrical main body and a proximal end face of the piston. The exterior cartridge cavity is destined to become deeper as the piston is displaced axially in the cartridge during use.

At least one of the contact surfaces may be axially offset from the other contact surfaces. If the axial position of one or more contact surfaces differs from the axial position of the remaining contact surfaces the axial force required to insert the first module part into the exterior cartridge cavity, past the narrowed rear entrance section, is reduced compared to a situation where all contact surfaces are arranged at the same axial position, simply because fewer contact surfaces need to be displaced by the circumferential bead at any one time during the axial movement of the sensor module.

For example, every other contact surface may be axially offset from its neighbouring contact surfaces.

In a particular embodiment of the invention the anti-rotation means comprises an equal number of radially outwardly projecting studs, where every other radially outwardly projecting stud forms a first group and the remaining radially outwardly projecting studs forms a second group, and where the respective contact surfaces of the first group are arranged at a first axial position and the respective contact surfaces of the second group are arranged at a second axial position offset from the first axial position.

When considering various factors in combination, such as the total rotational friction force, the axial friction force, the stability of the sensor module in the cartridge and the moulding process for producing the radially outwardly projecting studs, the inventors have determined that for many designs of the sensor module it is preferable that the anti-rotation means consists of 3-6 radially outwardly projecting studs.

In particular, the anti-rotation means may consist of a first pair of radially outwardly projecting studs and a second pair of radially outwardly projecting studs, and the studs of each of the first pair and the second pair may be arranged diametrically opposite from one another. In a preferred embodiment thereof the four radially outwardly projecting studs are equidistantly spaced.

The respective contact surfaces of the first pair of radially outwardly projecting studs may be axially offset from the respective contact surfaces of the second pair of radially outwardly projecting studs to thereby reduce the axial force required for introducing the first module part into the exterior cartridge cavity past the narrowed entrance section at the circumferential bead.

The sensor module may further comprise a module housing, a power source, such as e.g. a battery, and/or a processor. The sensor means may comprise a first sensor structure in, or in connection with, the first module part and a second sensor structure in, or in connection with, the second module part. The first sensor structure may e.g. comprise a transversal sensor surface axially and rotationally restricted or fixed with respect to the module housing, and the second sensor structure may e.g. comprise a plurality of flexibly supported and axially deflectable contact members adapted to sweep the transversal sensor surface in response to a relative rotational motion between the first module part and the second module part, thereby generating a plurality of signals, e.g. electrical signals, indicative of a relative angular displacement between the first sensor structure and the second sensor structure. The anti-rotation means is arranged to impede relative angular displacement between the first module part and the cartridge, which may otherwise potentially occur due to the torque exerted by the second sensor structure on the first sensor structure as the piston rod rotates and the contact members slide along the transversal sensor surface.

In an exemplary embodiment of the invention the transversal sensor surface comprises a plurality of electrically conductive sensor areas arranged in a pattern, and the contact members are adapted to sweep at least a subset of the plurality of electrically conductive sensor areas as the first sensor structure and the second sensor structure undergo relative rotation, thereby alternately connecting and disconnecting different sensor areas, a current connection being indicative of a current relative angular position of the first sensor structure and the second sensor structure. Electrical signals are thus generated for immediate processing in the processor which ultimately calculates the total relative angular displacement between the first sensor structure and the second sensor structure from the connections made, and on the basis thereof calculates a corresponding dose size, which is then e.g. presented on a visual display of the drug delivery device. Alternatively, the dose size may be calculated by an external device receiving data, e.g. wirelessly, from the sensor module.

In another aspect the invention provides a sensor module as described above in combination with a drug delivery device.

A drug delivery system is thereby provided comprising the sensor module and the drug delivery device. The sensor module may be pre-installed in the drug delivery device or supplied as a separate component for insertion into the drug delivery device.

The drug delivery device may be of the type where a threadedly supported piston rod is actuatable to pressurise a drug chamber, i.e. the drug delivery device may comprise a housing accommodating a dose expelling mechanism comprising a rotatable piston rod, and a cartridge rotationally fixed with respect to the housing, the cartridge comprising a drug chamber, defined by a portion of a cartridge wall, a distal self-sealing septum and a cartridge piston. In particular embodiments of the invention the drug delivery device then further comprises the sensor module arranged between the piston rod and the cartridge piston.

The sensor module may be arranged such that the first module part abuts or engages the cartridge piston and the second module part is rotationally fixed to the piston rod.

The cartridge wall comprises an interior surface which interfaces with the respective contact surfaces of the radially outwardly projecting studs during dose expelling. The contact surfaces may be radially inwardly displaceable against a bias force (e.g. because the radially outwardly projecting studs are radially compressible structures and/or are formed on pivotable levers), and the radial dimension of the first module part may be greater than the inner diameter of the cartridge wall such that the contact surfaces are displaced radially inwardly by the interior surface of the cartridge wall when the first module part is positioned in an exterior cartridge cavity defined by a portion of the cartridge wall and a proximal end surface of the cartridge piston, the contact surfaces thereby applying a radially outwardly directed force to the interior surface of the cartridge wall.

Hence, the radially outwardly projecting studs may be adapted to transition from an unstrained state to a strained state in response to an inwards displacement of the contact surfaces as the first module part enters the exterior cartridge cavity.

Since the drug delivery device may be shelved for a significant period of time before being taken into use it is undesirable to pre-install the sensor module in a position where the radially outwardly projecting studs are in the strained state, as this could lead to a gradual reduction of the contact force and resultantly to a gradual loss of friction in the interface between the cartridge wall and the contact surfaces. Several years of storage may result in a reduction of the contact force of up to about 30%. A drug delivery device which is shelved for a shorter period may lose markedly less friction and this may lead to an uncontrollable variance in a large batch of drug delivery devices.

Consequently, it is desirable to pre-install the sensor module in a position where the radially outwardly projecting studs are in the unstrained state. This means pre-installing the sensor module in a position where the cartridge wall does not support the first module part and therefore does not impede rotation thereof.

During transport and general handling of the drug delivery device the sensor module may be exposed to various jolting movements which may potentially cause the application of a torque thereto. If the first module part is unsupported this may lead to a slight angular displacement of the first module part relative to the second module part, which is rotationally locked to the fixed piston rod, and this may again lead to the sensor means being activated and detecting the slight angular displacement, both erroneously registering a dose expelling event and draining the power source.

To eliminate the risk of that happening the drug delivery device may further comprise a locking structure rotationally fixed with respect to the housing and adapted to engage with at least one of the radially outwardly projecting studs in a pre-use position of the sensor module relative to the housing. The locking structure may e.g. comprise an annular component fixed to the housing or an annular section of the housing, the annular component or section comprising a corrugated interior surface configured to axially receive and rotationally immobilise the first module part. In particular, the corrugated interior surface may comprise a plurality of axially enterable open compartments, or indentations, each configured to accommodate one of the radially outwardly projecting studs in an angularly fixed engagement.

The sensor module is then adapted to be moved axially relative to the housing, before the first dose expelling, from the pre-use position in which the contact surfaces are accommodated in the locking structure to an in-use position in which the contact surfaces are in contact with the interior surface of the cartridge wall.

Inferably, this movement may cause the radially outwardly projecting studs to transition from the unstrained state to the strained state. The axial force which must be applied by, or via, the dose expelling mechanism to move the sensor module from the pre-use position to the in-use position is thus larger than if there were no radially outwardly projecting studs because of the energy required to overcome the bias force resisting the radial displacement of the contact surfaces as the first module part enters the exterior cartridge cavity. This axial force may be reduced if at least one of the contact surfaces is axially offset from the other contact surfaces, as described above.

For the avoidance of any doubt, in the present context the term “drug” designates a medium which is used in the treatment, prevention or diagnosis of a condition, i.e. including a medium having a therapeutic or metabolic effect in the body. Further, the terms “distal” and “proximal” denote positions at or directions along a drug delivery device, or a needle unit, where “distal” refers to the drug outlet end and “proximal” refers to the end opposite the drug outlet end.

In the present specification, reference to a certain aspect or a certain embodiment (e.g. “an aspect”, “a first aspect”, “one embodiment”, “an exemplary embodiment”, or the like) signifies that a particular feature, structure, or characteristic described in connection with the respective aspect or embodiment is included in, or inherent of, at least that one aspect or embodiment of the invention, but not necessarily in/of all aspects or embodiments of the invention. It is emphasized, however, that any combination of the various features, structures and/or characteristics described in relation to the invention is encompassed by the invention unless expressly stated herein or clearly contradicted by context.

The use of any and all examples, or exemplary language (e.g., such as, etc.), in the text is intended to merely illuminate the invention and does not pose a limitation on the scope of the same, unless otherwise claimed. Further, no language or wording in the specification should be construed as indicating any non-claimed element as essential to the practice of the invention.

### BRIEF DESCRIPTION OF THE DRAWINGS

In the following the invention will be further described with references to the drawings, wherein

FIG. 1 shows a dose detection principle according to the prior art,

FIG. 2 is a perspective longitudinal section view of an injection device with an integrated dose sensing module according to an exemplary embodiment of the invention,

FIG. 3 is an exploded view of the dose sensing module,

FIG. 4 is a perspective longitudinal section view of the dose sensing module,

FIG. 5 is a side view of a wiper assembly used in the dose sensing module,

FIG. 6 is a distal perspective view of the wiper assembly,

FIGS. 7 and 8 are respective examples of alternative wiper assemblies for use in the dose sensing module,

FIG. 9 is a longitudinal section view of a dose sensing module according to another embodiment of the invention in a pre-use position outside a cartridge,

FIG. 10 shows the dose sensing module of FIG. 9 in an in-use position in the cartridge,

FIG. 11 is a cross-sectional view of the dose sensing module in the pre-use position, and

FIGS. 12a and 12b are respectively a perspective view and a side view of a part of a dose sensing module according to yet another embodiment of the invention.

In the figures like structures are mainly identified by like reference numerals.

### DESCRIPTION OF EXEMPLARY EMBODIMENTS

When/If relative expressions, such as “upper” and “lower”, “left” and “right”, “horizontal” and “vertical”, “clockwise” and “counter-clockwise”, etc., are used in the following, these refer to the appended figures and not necessarily to an actual situation of use. The shown figures are schematic representations for which reason the configuration of the different structures as well as their relative dimensions are intended to serve illustrative purposes only.

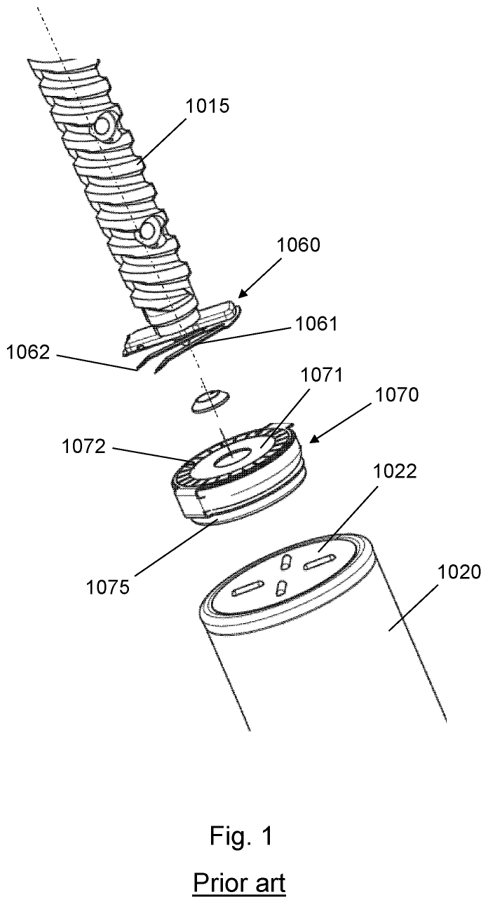

FIG. 1 shows a rotary sensor module according to the prior art, arranged between a distal end of a piston rod 1015 and a proximal end of a piston 1022 sealing a drug containing cartridge 1020. The sensor module, which is powered by a coin cell type battery 1075, comprises a first sensor part 1070 in the form of a flexible printed circuit board sheet having a proximally directed sensor surface 1071 on which 24 individual electrically conductive sensor areas 1072 are disposed circumferentially about a centre axis, and a second sensor part 1060 mounted on a distal end portion of the piston rod 1015 opposite the first sensor part 1070 and having contact structures in the form of two electrically connected flexible arms 1061, each terminating in a contact point 1062.

The first sensor part 1070 is adapted to engage, directly or indirectly, the piston 1022 such that no relative rotation therebetween is possible. The second sensor part 1060 is rotationally fixed to the piston rod 1015, and the contact points 1062 are adapted to engage and electrically connect various individual electrically conductive sensor areas 1072 upon relative rotational motion between the first sensor part 1070 and the second sensor part 1060, experienced as the piston rod 1015 rotates during a dose expelling action. This allows for an estimation of a total angular displacement exhibited by the piston rod 1015 during the dose expelling action and thereby of the amount of drug expelled.

During the dose expelling the piston rod 1015 undergoes a helical motion, and the axial component of this motion causes an axial advancement of the piston 1022 in the cartridge 1020, as the axial force from the piston rod 1015 is transferred to the proximal surface of the piston 1022 via the sensor module. In connection therewith the second sensor part 1060 is pressed against the first sensor part 1070 and this increases the contact pressure between the contact points 1062 and the sensor surface 1071, thereby reinforcing the electrical contact which generates the signal output. However, it also causes the flexible arms 1061 to deflect against the axial direction of travel of the piston rod 1015, whereby elastic energy is stored therein.

In the course of the dose expelling the flexible arms 1061 remain so deflected, but when the piston rod 1015 eventually stops and the whole dose expelling system relaxes the elastic energy stored in the flexible arms 1061 is released and transferred to the sensor surface 1071 which is urged axially away from the second sensor part 1060.

The additional axial movement of the first sensor part 1070 causes an additional axial movement of the piston 1022 which in turn causes a small additional dose to be expelled. Notably, this additional dose is expelled after the piston rod 1015 has stopped its movement and will resultantly require the user to wait a little longer before removing the injection needle from the skin in order to ensure that the entire dose has been received. Furthermore, even though it is advantageous that an increased contact pressure reduces the risk of an accidental loss of contact between the contact points 1062 and the sensor surface 1071 it comes with the cost of an increased friction in the rotational interface between the first sensor part 1070 and the second sensor part 1060, which increases the risk that an angular displacement is introduced to the first sensor part 1070, thereby affecting the accuracy of the dose detection principle.

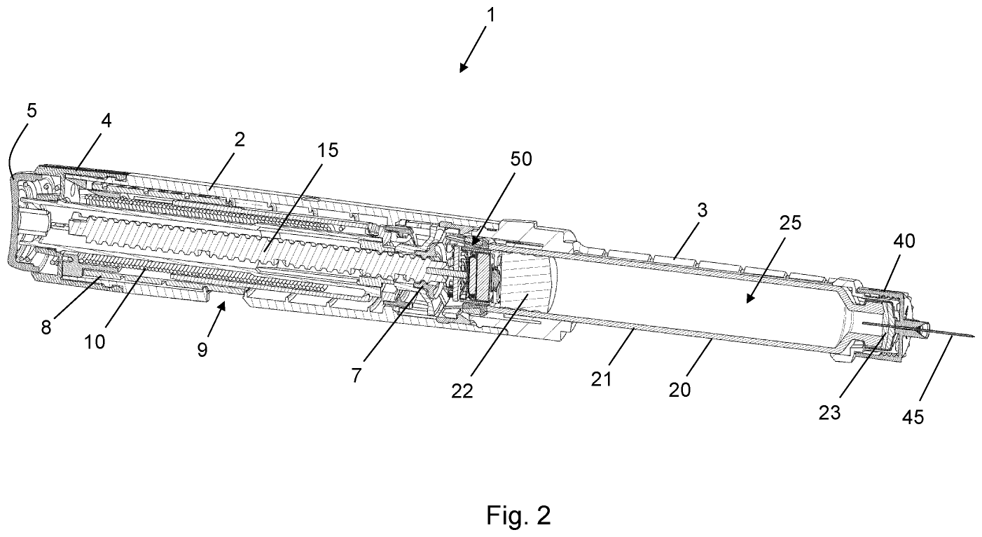

FIG. 2 is a perspective longitudinal section view of an injection device 1 having an integrated sensor module 50 according to an exemplary embodiment of the invention. The injection device 1 is of the prefilled autopen injector type, with an elongated housing 2 extending along a reference axis and accommodating a dose expelling mechanism. A cartridge holder 3, holding a cartridge 20 with an interior chamber 25 defined by a cartridge wall 21, a distal penetrable septum 23 and a proximal piston 22, is permanently fixed to the housing 2. The chamber 25 is at least substantially filled with a liquid substance (not visible). In the depicted state of the injection device 1 a needle assembly 40 is attached to a needle mount portion of the cartridge holder 3 in such a manner that an injection needle 45 has penetrated the septum 23 to establish fluid communication to the chamber 25.

A user operable dose dial 4 is arranged at a proximal end portion of the housing 2 for selective setting of a dose to be ejected from the cartridge 20. The dose dial 4 is operatively coupled with a scale drum 8 which displays a selected dose through a window 9. An injection button 5 is axially depressible to release a windable torsion spring 10. The release of the torsion spring 10 will cause a helical advancement of a piston rod 15 through a nut member 7 in the housing 2 and thereby result in an execution of a dose expelling action.

Details of the dose setting and the dose expelling mechanisms are irrelevant to the present invention and will accordingly not be provided in the present text. For an example of how such mechanisms may be constructed reference is made to WO 2015/071354, particularly p. 10, l. 21-p. 15, l. 13. What is important is that the rotational movement of the piston rod 15 during dose expelling is correlated with the prompted movement of the piston 22 through the design of the piston rod thread and the nut member 7 such that a predetermined angular displacement of the piston rod 15 relative to the housing 2 corresponds to a predetermined axial displacement of the piston 22 relative to the cartridge wall 21. This relationship may in principle be chosen arbitrarily by the manufacturer, with a view to the dimensions of the cartridge 20. In the present example a 15° angular displacement of the piston rod corresponds to a specific axial displacement of the piston 22 which results in the expelling of 1 IU of the contained substance through the injection needle 45.

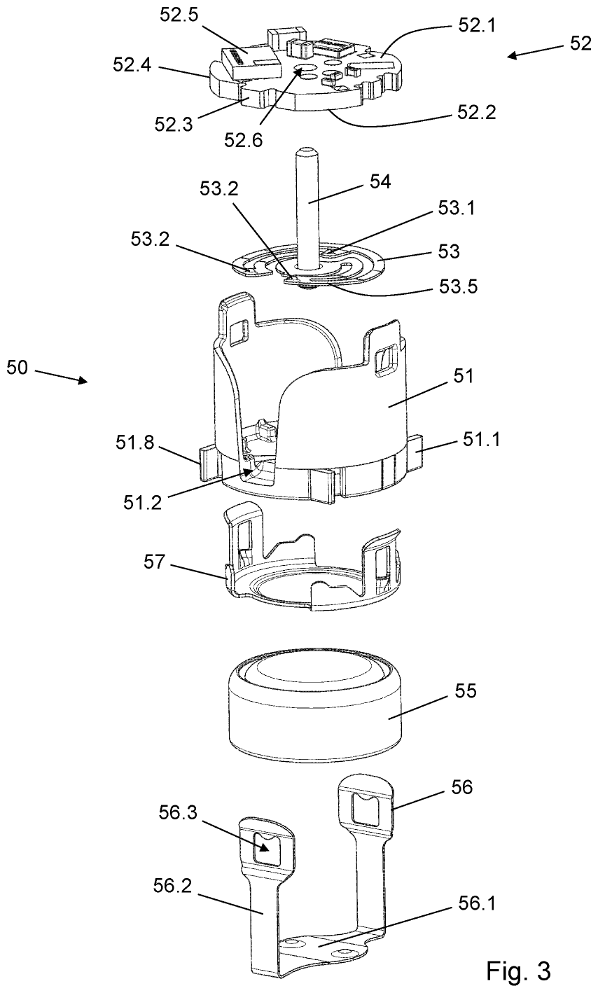

FIG. 3 is an exploded view highlighting the individual elements of the present sensor module 50. The sensor module 50 comprises a first sensor part in the form of a PCB assembly 52 with a rigid support sheet 52.4 having a proximal surface 52.1 carrying various electronic components 52.5, including a processor, and a distal surface 52.2 carrying a plurality of electrically conductive sensor areas (not visible), the configuration of which will be described below. The support sheet 52.4 has an overall circular periphery, but is provided with several notches, some of which resulting in a pair of diametrically opposite radial protrusions 52.3. Furthermore, the support sheet 52.4 has a central through-going bore 52.6.

The first sensor part is complemented by a second sensor part in the form of a wiper 53 being fixedly mounted to a piston rod connector 54 to ensure joint rotation therewith. The piston rod connector 54 extends axially through the through-going bore 52.6 and is adapted for press-fit engagement with a cavity in a distal end portion of the piston rod 15, as shown on FIG. 2. This provides for a joint movement of the piston rod 15 and the piston rod connector 54. The wiper 53 comprises one ground contact 53.1 and two code contacts 53.2 arranged on respective flexible arms 53.5 and adapted to galvanically connect with the electrically conductive sensor areas on the distal surface 52.2 of the support sheet 52.4, as described in more detail below. Notably, the ground contact 53.1 and the code contacts 53.2 are all proximally directed.

The two sensor parts, forming a rotary encoder system, are accommodated in a module housing 51 which also accommodates a power source in the form of a battery 55, a retainer 56 also functioning as a positive battery connector, and a rigid (negative) battery connector 57. The retainer 56 has a transversal support surface 56.1 for carrying the battery 55 and two axially extending opposite retainer arms 56.2. Each retainer arm 56.2 is provided with a proximal cut-out 56.3 shaped to receive one of the radial protrusions 52.3, thereby rotationally interlocking the retainer 56 and the PCB assembly 52 and axially restricting the support sheet 52.4. The module housing 51 has a pair of diametrically opposite side openings 51.2 shaped to receive the retainer arms 56.2 so as to rotationally interlock, or at least substantially rotationally interlock, the retainer 56 and the module housing 51, and a plurality of anti-rotation tabs 51.1 spaced apart along its circumference, each anti-rotation tab 51.1 comprising a contact surface 51.8 for interaction with an interior surface of the cartridge wall 21. The PCB assembly 52 is thus at least substantially rotationally locked with respect to the module housing 51, which in turn is rotationally frictionally fitted in the cartridge 20, which is rotationally fixed in the cartridge holder 3. The PCB assembly 52 is thereby at least substantially rotationally fixed with respect to the housing 2 and accordingly suitable as reference component for measuring angular displacements of the piston rod 15.

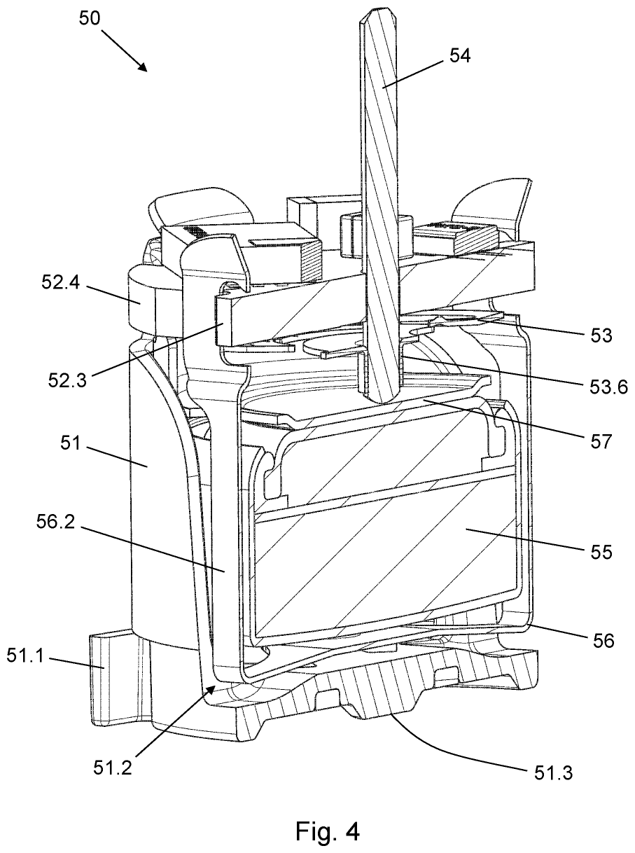

FIG. 4 is a perspective longitudinal section view of the sensor module 50 in an assembled state. As can be seen the piston rod connector 54 extends through the through-going bore 52.6 in the support sheet 52.4 and is press-fitted with a sleeve 53.6 on the wiper 53. The module housing 51 has a foot 51.3 which rests against the piston 22 (cf. FIG. 2). Furthermore, the figure shows the position of the retainer arms 56.2 in the side openings 51.2 and the arrangement of the radial protrusions 52.3 in the cut-outs 56.3. During a dose expelling action with the injection device 1 the rotation of the piston rod 15 is transferred to the piston rod connector 54 and further on to the wiper 53. The ground contact 53.1 and the code contacts 53.2 thus sweep the sensor areas of the distal surface 52.2 which remains, at least substantially, rotationally stationary due to the engagement between the radial protrusions 52.3 and the cut-outs 56, the fitting of the retainer arms 56.2 in the side openings 51.2, the frictional interface between the foot 51.3 and the piston 22, and the frictional interface between the anti-rotation tabs 51.1 and the cartridge wall 21.

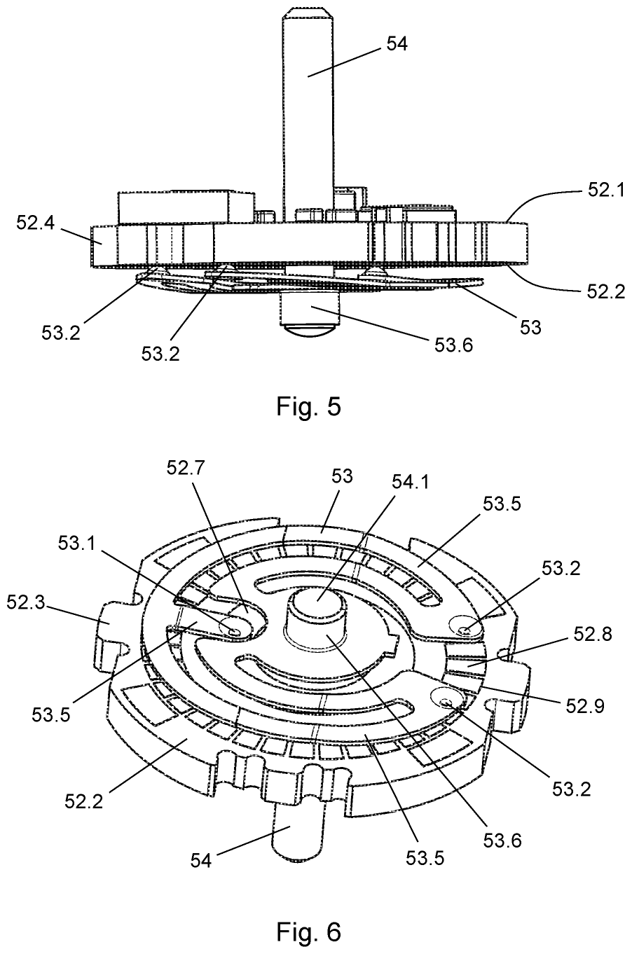

FIG. 5 is a side view of the two sensor parts showing the connection between the ground contact 53.1 and the code contacts 53.2 and the distal surface 52.2 of the support sheet 52.4, and FIG. 6 is a perspective distal view of the same. In the shown exemplary embodiment of the invention the aforementioned plurality of electrically conductive sensor areas on the distal surface 52.2 are arranged such that a single circular ground track 52.7 provides a ground connection for the ground contact 53.1 and 36 individual code fields 52.8 together constitute a code track 52.9 which the code contacts 53.2 are adapted to sweep. A secondary ground connection is provided through a spherical end 54.1 of the piston rod connector 54 contacting the (negative) battery connector 57. The secondary ground connection may be relevant to stabilise the signal output in case the dynamics of the dose expelling mechanism generates vibrations in the sensor module 50.

As the piston rod connector 54 rotates jointly with the piston rod 15 during a dose expelling action the two code contacts 53.2, which are circumferentially separated by 45°, respectively sweep the code track 52.9, generating signals representative of the angular position of the wiper 53 as different code fields 52.8 get connected to ground. The two sensor parts output a 4-bit Gray code, i.e. eight different codes which for a 360° rotation of the wiper 53 are repeated nine times, giving 72 distinguishing codes. This output thus forms the basis for an estimation, by one or more of the electronic components 52.5 including the processor, of the total angular displacement of the piston rod 15 during a dose expelling action, and thereby for an estimation of the expelled dose.

For galvanic sensors like the herein described it is crucial that the contact pressure on each physical contact is sufficiently high to ensure a stable signal. This prerequisite is met by the design of the present sensor module 50, where the combination of the flexible arms 53.5 and the sleeve 53.6 and the restricted axial play of the radial protrusions 52.3 in the cut-outs 56.3 enables an arrangement of the wiper 53 on the piston rod connector 54 relative to the support sheet 52.4 which provide a spring reinforced contact between the ground contact 53.1 and the ground track 52.7 as well as between the respective code contacts 53.2 and the code track 52.9. However, importantly, the fact that the wiper 53 is positioned distally of the support sheet 52.4 such that the flexible arms 53.5 are deflected distally and the respective ground and code contacts 53.1, 53.2 thereby provide proximally directed forces to the support sheet 52.4 is advantageous because during a dose expelling action when the piston rod connector 54 applies an axially directed force to the battery connector 57 this will not result in a further deflection of the flexible arms 53.5 as the wiper 53 is not pressed against the support sheet 52.4, i.e. no additional elastic energy is stored in the flexible arms 53.5 which needs to be released during the subsequent relaxation of the dose expelling system, and the problem of prolonged dose expelling is thus solved.

Furthermore, since the wiper 53 is not being pressed against the support sheet 52.4 as a result of the advancing piston rod connector 54 the contact pressure in the respective ground contact 53.1/ground track 52.7 and code contact 53.2/code track 52.9 interfaces is not increased during dose delivery. The friction in the rotational interface between the two sensor parts is therefore also not increased, which means that the torque applied by the wiper 53 to the support sheet 52.4 is not increased. The risk of angular displacement of the support sheet 52.4 against the rotation prevention mechanism provided by the interaction between the anti-rotation tabs 51.1 and the cartridge wall 21 is resultantly reduced compared to a solution, e.g. like the one shown in FIG. 1, where the flexible arms exhibit further deflection during piston rod advancement.

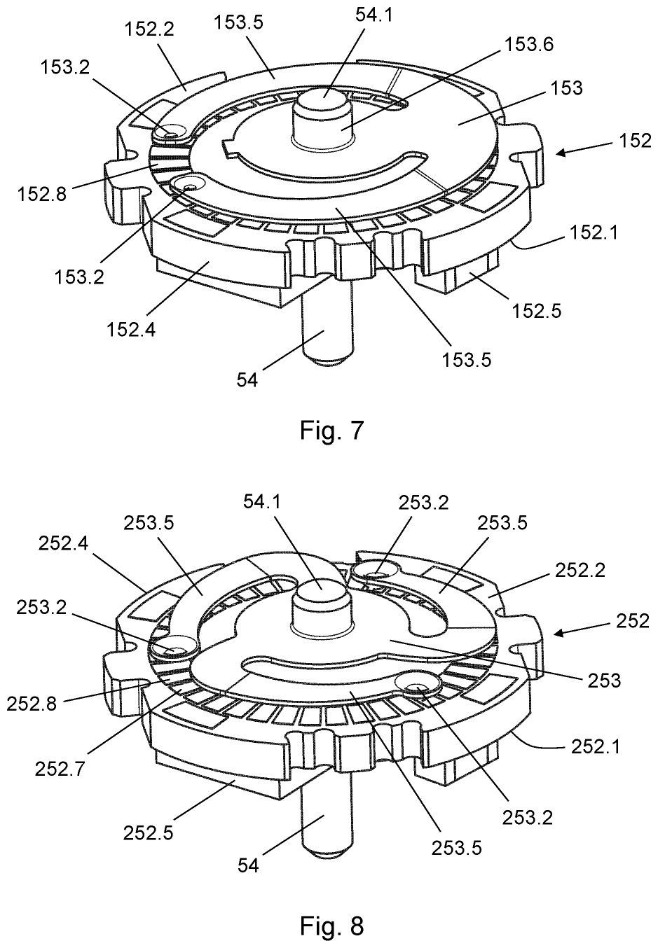

FIG. 7 is a perspective distal view of two sensor parts of an alternative rotary encoder system used in a sensor module according to another embodiment of the invention. The sensor parts comprise a wiper 153 and a PCB assembly 152 held in mutual position by the piston rod connector 54 in a manner similar to that disclosed in connection with the previous embodiment. The geometrical configuration of the PCB assembly 152 as well as its interaction with other components of the sensor module is identical to that of the formerly described PCB assembly 52. Particularly, the PCB assembly 152 comprises a rigid support sheet 152.4 having a proximal surface 152.1 which carries various electronic components 152.5, including a processor, and a distal surface 152.2 on which is disposed a plurality of electrically conductive code fields 152.8 arranged side by side to thereby provide a circular code track. However, contrary to the former embodiment the distal surface 152.2 does not comprise a dedicated ground track. Instead, the ground connection is supplied via the spherical end 54.1 of the piston rod connector 54 being in contact with the (negative) battery connector 57, similarly to the above described.

The wiper 153 comprises a sleeve 153.6 press-fitted onto the piston rod connector 54, to ensure joint rotation of the piston rod 15 and the wiper 153, and two code contacts 153.2, each arranged at an end portion of a flexible arm 153.5 capable of axial deflection. The code contacts 153.2 are angularly separated by 45° and will when rotated relative to the distal surface 152.2 respectively sweep the code fields 152.8 and produce a 4-bit Gray code, similarly to the previous embodiment. The fact that only two wiper contacts sweep the distal surface 152.2 provides for a reduced internal friction and therefore a reduced torque between the two sensor parts, compared to three sweeping contacts. Hence, the risk of angular displacement of the PCB assembly 152 against the rotation prevention mechanism provided by the interaction between the anti-rotation tabs 51.1 and the cartridge wall 21 is reduced even further, while the advantageous containment of the forces from the flexible arms 153.5 between the PCB assembly 152 and the battery 55 is still obtained, eliminating the prolonged dose expelling problem.

FIG. 8 is a perspective distal view of two sensor parts of another alternative rotary encoder system used in a sensor module according to a third embodiment of the invention. Similarly to the previous embodiments the sensor parts comprise a wiper 253 and a PCB assembly 252 held in mutual position by the piston rod connector 54. The geometrical configuration of the PCB assembly 252 as well as its interaction with other components of the sensor module is identical to that of the formerly described PCB assembly 52. Particularly, the PCB assembly 252 comprises a rigid support sheet 252.4 having a proximal surface 252.1 which carries various electronic components 252.5, including a processor, and a distal surface 252.2 on which is disposed a plurality of electrically conductive sensor areas.

However, contrary to the former embodiments the distal surface 252.2 carries 40 electrically conductive sensor areas arranged in a circular track pattern where every other sensor area constitutes a ground field 252.7 and every other sensor area constitutes a code field 252.8. A secondary ground connection is supplied via the spherical end 54.1 of the piston rod connector 54 being in contact with the (negative) battery connector 57, as described above in connection with the first embodiment of the invention.

A wiper 253 is attached to the piston rod connector 54 and is adapted to sweep the 40 electrically conductive sensor areas as the piston rod 15 rotates during a dose expelling action (as described above). The wiper 253 has three flexible arms 253.5, each terminating in a contact point 253.2 which is adapted to galvanically connect with a ground field 252.7 or a code field 252.8, depending on the angular position of the wiper 253 relative to the PCB assembly 252. The three contact points 253.2 are separated 120° from each other such that one contact point 253.2 is always connected to a ground field 252.7 and two contact points 253.2 are always connected to a code field 253.8. The two sensor parts output a 4-bit Gray code and offer a higher resolution than the former two embodiments of the invention, enabling an even more accurate estimation of the total relative angular displacement between the PCB assembly 252 and the wiper 253, and thereby of the total angular displacement of the piston rod 15 relative to the housing 2, during a dose expelling event.

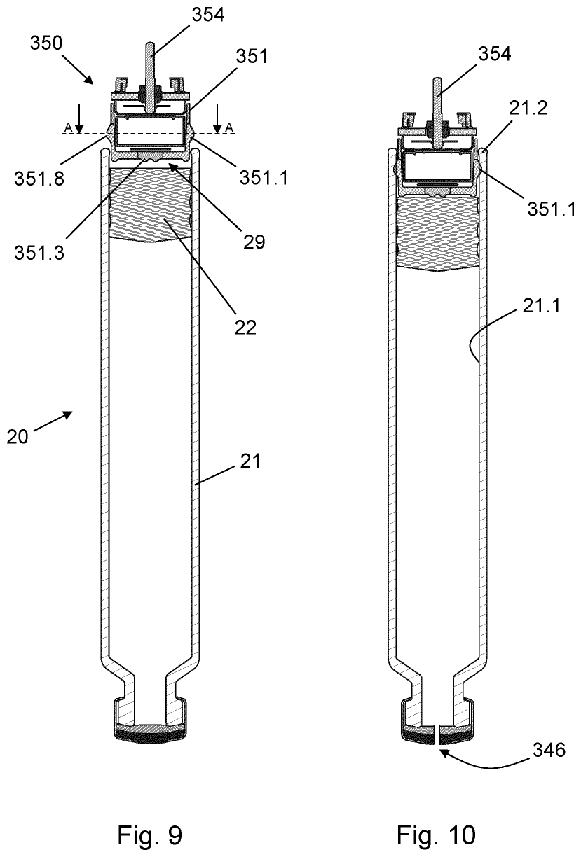

FIG. 9 is a longitudinal section view of a sensor module 350 according to a fourth embodiment of the invention, in a pre-use position outside the cartridge 20. The rest of the injection device is omitted from the view for the sake of clarity. The structure of the sensor module 350 resembles that of the sensor module 50 described with respect to the first embodiment. Accordingly, the sensor module 350 comprises a module housing 351 with a foot 351.3 for engagement with the piston 22, and a piston rod connector 354 for engagement with the piston rod (not shown). The main difference vis-à-vis the former sensor module 50 is that the module housing 351 comprises a pair of anti-rotation tabs 351.1 with respective contact surfaces 351.8 which are arranged more proximally than the contact surfaces 51.8 of the module housing 51.

The sensor module 350 is adapted to be displaced axially, during the first use of the injection device, from the pre-use position in which it is spaced apart from the piston 22 to an in-use position in an exterior cartridge cavity 29 defined by a proximal end portion of the cartridge wall 21 and a proximal end face of the piston 22. During this displacement from the pre-use position to the in-use position the anti-rotation tabs 351.1 will be deflected radially inwardly against a bias force provided by the structure of the module housing 351, and the sensor module 350 accordingly transitions from an unstrained state to a strained state.

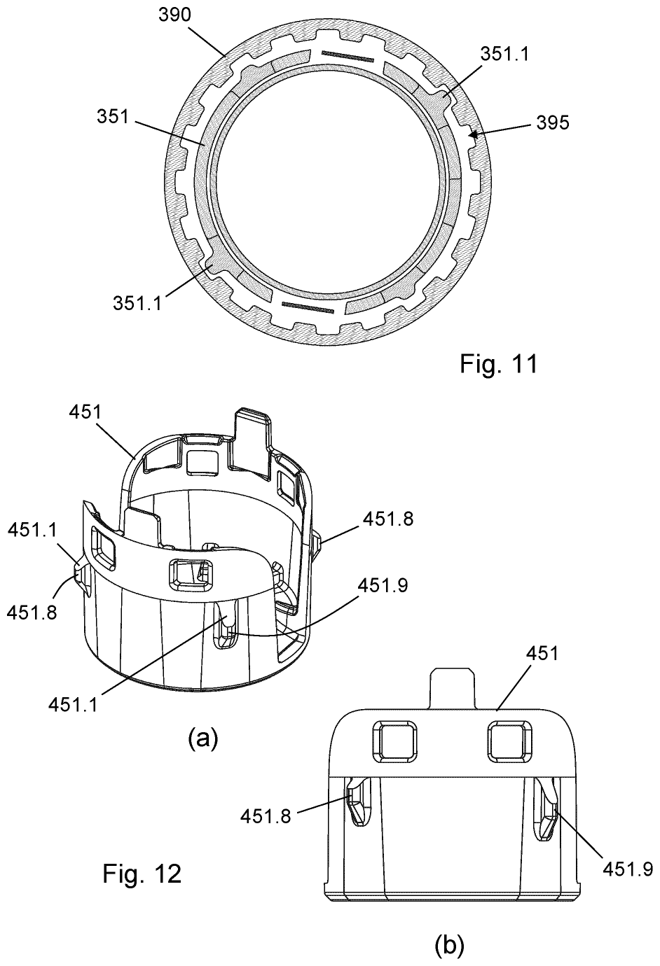

In the shown pre-use position of the sensor module 350 the piston rod connector 354 is prevented from rotating about a longitudinal reference axis, because the piston rod is rotationally fixed with respect to the injection device housing in a pre-use state of the injection device. Furthermore, the module housing 351 is prevented from rotating because the anti-rotation tabs 351.1 engage with a locking ring 390. Said locking ring 390 is not shown in FIG. 9, but can be seen in FIG. 11 which is a cross-sectional view through section A-A. The locking ring 390 is rotationally fixed with respect to the injection device housing and has an interior corrugated surface which forms a plurality of radial indentations 395, each configured to axially receive one of the anti-rotation tabs 351.1 so as to provide a rotationally interlocked connection to the module housing 351.

The sensor module 350 is thus rotationally fixed in a pre-use state of the injection device, so even if the injection device is dropped on the ground or otherwise exhibits jolting movements, e.g. in connection with transportation or general handling, there is no risk of inadvertently wakening the sensor electronics and thereby draining the battery.

FIG. 10 shows the sensor module 350 in an in-use position where the module housing 351 has been moved by the piston rod (not shown) into the exterior cartridge cavity 29, which has deepened due to a resultant displacement of the piston 22 and ejection of a volume of drug through a channel 346 in the cartridge septum provided by an inserted injection needle (not shown). In the in-use position the contact surfaces 351.8 interface with an interior surface 21.1 of the cartridge wall 21.

During movement of the sensor module 350 to this position the anti-rotation tabs 351.1 pass a circumferential bead 21.2 at the proximal end of the cartridge 20, and the narrowed entrance section provided by the circumferential bead 21.2 gives rise to a local increase in the axial force profile for the piston rod. Once the anti-rotation tabs 351.1 have passed the circumferential bead 21.2 the contact surfaces 351.8 will apply a radially outwardly directed force to the interior surface 21.1 of the cartridge wall 21 and thereby serve to impede rotation of the module housing 351 relative to the cartridge 20.

FIG. 12a is a perspective view of a module housing 451 of a sensor module according to a fifth embodiment of the invention, which sensor module can be used in the injection device with the cartridge 20 as an alternative to the previously described sensor module 350.

The module housing 451 carries exactly four anti-rotation tabs 451.1, formed with equidistant spacing along its circumference. The anti-rotation tabs 451.1 are arranged as two pairs of diametrically opposite protrusions, where a first pair has first contact surfaces 451.8, and a second pair has second contact surfaces 451.9 which are axially offset from the first contact surfaces 451.8. The different axial positions of first contact surfaces 451.8 and the second contact surfaces 451.9 are more clearly depicted in FIG. 12b, which is a side view of the module housing 451. Each anti-rotation tab 451.1 is radially compressible against a bias force provided by its form and constituent material.

During use, when the sensor module with the module housing 451 is pressed through the narrowed entrance section of the cartridge 20 and into the exterior cartridge cavity 29 the second contact surfaces 451.9 will be urged radially inwardly first, followed by the first contact surfaces 451.8. The local increase in the axial force profile for the piston rod experienced during the insertion of the module housing 451 into the exterior cartridge cavity 29 is thus smaller than it would be if all four anti-rotation tabs 451.1 had the same axial position and would have to be urged radially inwardly at the same time. This markedly reduces the maximum force required to move the sensor module from the pre-use position to the in-use position, and thus provides for a smoother insertion of the sensor module into the exterior cartridge cavity 29.

1. A sensor module adapted to be arranged in a cartridge based drug delivery device between a rotatable piston rod and a cartridge piston for capturing dose data from a dose expelling event, the sensor module extending along a reference axis and comprising:
a first module part adapted to engage the cartridge piston and comprising anti-rotation means for establishing a frictional interface to the cartridge to impede rotation of the first module part relative thereto,
a second module part adapted to engage the rotatable piston rod, and
sensor means adapted to detect an extent of relative rotational motion between the first module part and the second module part,
the anti-rotation means comprises a plurality of radially outwardly projecting studs, each comprising a contact surface adapted to establish frictional contact with an interior surface of the cartridge.

2. The sensor module according to claim 1, wherein the radially outwardly projecting studs are circumferentially spaced apart along an annular outer surface of the first module part.

3. The sensor module according to claim 2, wherein the radially outwardly projecting studs are equidistantly spaced apart.

4. The sensor module according to claim 2, wherein at least one of the contact surfaces is axially offset from the other contact surfaces.

5. The sensor module according to claim 4, wherein the anti-rotation means comprises an equal number of radially outwardly projecting studs, wherein every other radially outwardly projecting stud forms a first group and the remaining radially outwardly projecting studs forms a second group, and wherein the respective contact surfaces of the first group are arranged at a first axial position and the respective contact surfaces of the second group are arranged at a second axial position offset from the first axial position.

6. The sensor module according to claim 4, wherein the anti-rotation means consists of 3-6 radially outwardly projecting studs.

7. The sensor module according to claim 4, wherein the anti-rotation means consists of a first pair of radially outwardly projecting studs and a second pair of radially outwardly projecting studs, the studs of each of the first pair and the second pair being arranged diametrically opposite from one another.

8. The sensor module according to claim 7, wherein the respective contact surfaces of the first pair of radially outwardly projecting studs are axially offset from the respective contact surfaces of the second pair of radially outwardly projecting studs.

9. The sensor module according to claim 1, wherein the contact surfaces are radially inwardly displaceable against a bias force.

10. The sensor module according to claim 1 in combination with a drug delivery device comprising:
a housing accommodating a dose expelling mechanism comprising a piston rod, and
a cartridge rotationally fixed with respect to the housing, the cartridge comprising a drug chamber, defined by a cartridge wall and sealed distally by a self-sealing septum and proximally by a cartridge piston.

11. The sensor module and drug delivery device according to claim 10, further comprising a locking structure rotationally fixed with respect to the housing, wherein the sensor module is adapted to be moved axially relative to the housing, before the first dose expelling, from a pre-use position in which at least one of the radially outwardly projecting studs is engaged with the locking structure to an in-use position in which the contact surfaces are in contact with an interior surface of the cartridge wall.

12. The sensor module and drug delivery device according to claim 11, wherein the contact surfaces are radially inwardly displaceable against a bias force, and wherein the radially outwardly projecting studs are adapted to transition from an unstrained state to a strained state in response to an inwards displacement of the contact surfaces as the sensor module is moved from the pre-use position to the in-use position, each contact surface thereby applying a radial force to the interior surface of the cartridge wall.
